(19) United States
(12) Patent Application Publication
(10) Pub. No.: US 2021/0196110 A1
(43) Pub. Date: Jul. 1, 2021

(54) ELECTRONIC VIDEO LASER ENDOSCOPE

(71) Applicant: Martin Uram, Little Silver, NJ (US)

(72) Inventor: Martin Uram, Little Silver, NJ (US)

(21) Appl. No.: 17/138,367

(22) Filed: Dec. 30, 2020

Related U.S. Application Data
(60) Provisional application No. 62/955,707, filed on Dec. 31, 2019.

(51) Int. Cl. A61B 1/05; A61B 1/06; A61B 1/00

(52) U.S. Cl. CPC A61B 1/051; A61B 1/063; A61B 1/00009; A61B 1/00016; A61B 1/00167; A61B 1/0676

(57) ABSTRACT

Electronic laser video endoscope comprising laser output, illumination output and imaging component disposed at a distal end of a probe, or a cannula, of the endoscope to facilitate precise illumination, targeted laser energy delivery, and accurate imaging at the site of a procedure, where the imaging component comprises an electronic image detection module. The electronic image detection module comprises an image sensor, such as CMOS (complementary metal-oxide semiconductor) and/or CCD (charge-coupled device) image sensor, disposed at a distal end of the probe.

### CROSS-REFERENCE TO RELATED APPLICATIONS

This application claims priority to U.S. Provisional Patent Application No. 62/955,707, filed Dec. 31, 2019, which is hereby incorporated by reference in its entirety.

### BACKGROUND

### 1. General Field of Disclosure

This disclosure relates in general to a medical laser video endoscope and more particularly to one in which the operating probe includes imaging, illumination, and lasing components.

### 2. Description of Related Art

Laser video endoscopes are used in glaucoma, retinal and vitrectomy operations.

Conventional endoscopes employ a probe typically passing through a 20 gauge tissue incision during ophthalmological surgery. A 20 gauge incision has been a standard in the art of ophthalmological surgery and is used for entry by instruments employed during an ophthalmological surgical routine. A smaller 23 gauge sleeve, such as a trocar sleeve which is a tube implanted in a body wall which permits insertion and removal of a surgical instrument without touching the body wall tissue, is also known in conventional applications for smaller incisions. Accordingly, endoscopes with probes having a smaller diameter can be used to fit through the 23 gauge sleeve.

Laser video endoscope comprising a laser guide, an illumination guide and an image guide, which can be fiber optical guides extending through a probe portions of the endoscope and through a hand piece that supports the probe portion which can protrude from the distal end of the hand piece, are described in prior U.S. Pat. Nos. 5,121,740, 6,997,868, and 10,226,167, as well as U.S. Pub. No. US 2011-0282139, U.S. Pub. No. 2012-0265010 and U.S. Pub. No. US 2016-0095507, the entire disclosures of all of which are incorporated herein by reference.

### SUMMARY OF THE DISCLOSURE

Exemplary embodiments of the present disclosure provide an electronic laser video endoscope comprising a laser output, an illumination output and an imaging component disposed at a distal end of a probe, or a cannula, of the endoscope to facilitate precise illumination, targeted laser energy delivery, and accurate imaging at the site of a procedure, where the imaging component comprises an electronic image detection module.

According to an exemplary implementation of the embodiments of the present disclosure, the electronic image detection module comprises an image sensor, such as a CMOS (complementary metal-oxide semiconductor) and/or CCD (charge-coupled device) image sensor, disposed at a distal end of the probe.

According to another exemplary implementation of the embodiments of the present disclosure, the electronic image detection module consists of a CMOS, or a CCD, image sensor disposed at the distal end of the probe with wired and/or wireless connection of the image sensor to an image processing or storage module.

According to an exemplary implementation of the embodiments of the present disclosure, the endoscope optionally include a hand piece with the probe extending distally from a distal end of the hand piece, such that proximal endo of the probe is attached to the hand piece and the distal end of the probe is disposed closest to the site of the procedure during operation.

According to a further exemplary implementation of the embodiments of the present disclosure, one or more optical and/or electronic connections extend through the probe from the distal end of the probe to at least the proximal end of the probe to facilitate the laser output, the illumination output and connection to the imaging component.

According to yet further exemplary implementation of the embodiments of the present disclosure, optionally at least one of the connections comprises a laser guide, such as an optical fiber, extending from the distal end of the probe to a source of laser energy (or a laser source).

According to still further exemplary implementation of the embodiments of the present disclosure, optionally at least one of the connections comprises an illumination guide, such as an optical fiber or a bundle of fibers, extending from the distal end of the probe to a source of illumination (or a light source).

According to yet another exemplary implementation of the embodiments of the present disclosure, optionally at least one of the connections comprises an electronic connection, such as a conductive wire or wires, extending from the distal end of the probe to an image and/or video processing and/or storage and/or transmission unit.

According to still another exemplary implementation of the embodiments of the present disclosure, a distal portion of a probe of a laser video endoscope includes: a laser guide comprising a laser fiber disposed within the inner diameter of the distal portion of the probe; an image module comprising a CMOS or a CCD image sensor, disposed within the inner diameter of the distal portion of the probe not occupied by the laser fiber; and an illumination guide comprising an illumination bundle, which has a plurality of fibers filling the remaining of the inner diameter of the distal portion of the probe not occupied by the laser fiber and the image module.

Exemplary embodiments of the present disclosure provide endoscope designs where a laser fiber can selectively accommodate input from laser energy sources having different wavelengths, such as for example a green laser having a wavelength of 532 nanometers.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 is a schematic illustration of an endoscope system according to an exemplary embodiment of the present disclosure.

FIG. 2 is a front view of a distal end of a probe of an endoscope system according to an exemplary embodiment of the present disclosure.

FIG. 3 is a side cross-sectional view of a distal end of a probe of an endoscope system according to an exemplary embodiment of the present disclosure.

FIGS. 4 is a block diagram illustration of an endoscope system according to an exemplary embodiment of the present disclosure.

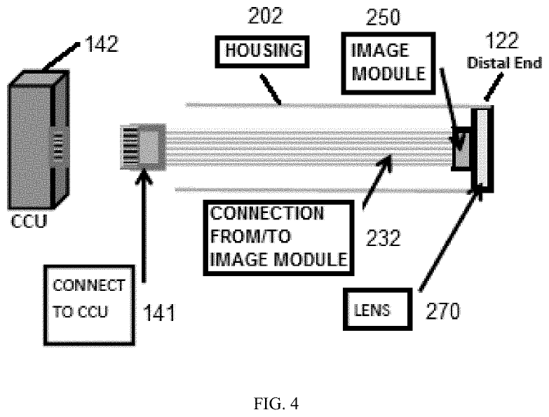

### DETAILED DESCRIPTION OF ILLUSTRATIVE EMBODIMENTS

The matters defined in the description such as a detailed construction and elements are nothing but the ones provided to assist in a comprehensive understanding of the disclosure. Accordingly, those of ordinary skill in the art will recognize that various changes and modifications of the embodiments described herein can be made without departing from the scope and spirit of the disclosure. Also, well-known functions or constructions are omitted for clarity and conciseness. Certain exemplary embodiments of the present disclosure may be described below in the context of commercial application. Such exemplary implementations are not intended to limit the scope of the present disclosure, which is defined in the appended claims.

It is to be noted that, while descriptive terms such as “hand piece”, “probe”, “fiber”, “wire”, “connection”, “optical”, “electrical”, “module”, “image”, and “unit” are used throughout this specification, it is not intended to limit components that can be used in combinations or individually to implement various aspects of the embodiments of the present disclosure.

Referring now to the drawings, wherein like reference numerals designate identical or corresponding parts throughout the several views, embodiments of the present disclosure are shown in schematic detail.

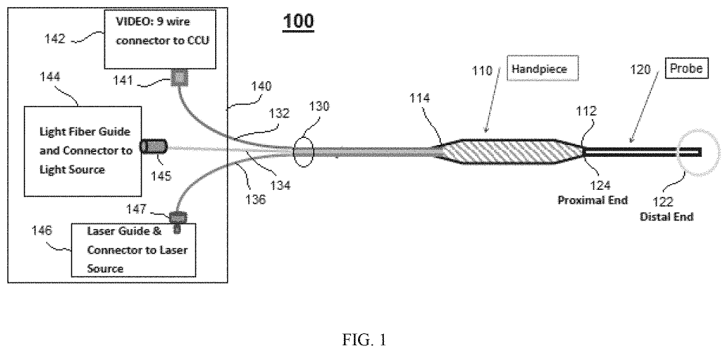

FIG. 1 is schematic diagram of an endoscope system 100 according to an exemplary embodiment of the disclosure. As illustrated in FIG. 1 endoscope system 100 comprises an operating probe 120, a hand piece 110, and interface 130 which comprises a connection, such as an optical connection—for example a laser guide, 136, another connection, such as an optical connection—for example a fiber bundle, 134, and yet another connection, such as an electrical or electronic connection—for example a wire, 132. These connections 132, 134, 136 extend through the probe 120, from the distal end 122 to the proximal end 124 of the probe 120, to respective terminals of an image/video processing/storage/transmittal unit 142—for example a CCU (camera control unit), a light source unit 144, and a laser source unit 146.

In an exemplary implementation, proximal end 124 of the probe 120 is attached to distal end of hand piece 110, the probe 120 extending distally from hand piece 110.

In yet another exemplary implementation, connections 132, 134, 136 of interface 130 extend from proximal end 124 of probe 120 through hand piece 110, from the distal end 112 to the proximal end 114 of the hand piece 120, to respective terminals of an image processing/storage/transmittal unit 142, light source 144, and laser source (or source of laser energy) 146 via respective connectors 141, 145, 147.

According to exemplary implementations, image processing/storage/transmittal unit 142, light source unit 144, and laser source unit 146 can be configured as individual components, modules or modular components, or within a base module 140.

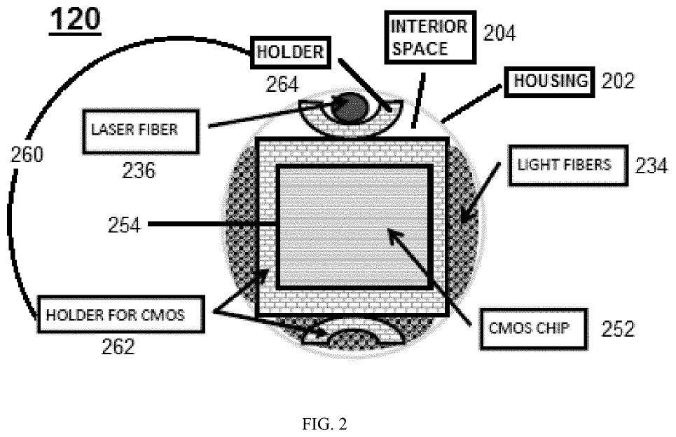

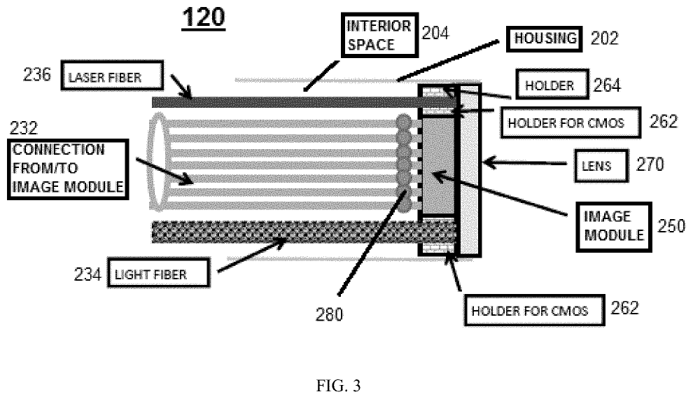

Referring to FIGS. 2 and 3, probe 120 of an electronic video laser endoscope according to an exemplary embodiment of the present disclosure comprises a housing 202—for example in a shape of a cannula—having an interior space 204 which extends from distal end 122 to proximal end 124 of the probe 120. An image module 250 is disposed at a distal end 122 of probe 120 and includes an image capturing element 252 comprising, or consisting exclusively of, a CMOS or a CCD imaging chip.

In an exemplary implementation, a support 260 for image module 250 can be provided at distal end 122. Support 260 may optionally include a CMOS or CCD chip holder 262 and/or holder 264—for example for a laser fiber 236.

According to exemplary implementations of the embodiments of the disclosure, image capturing element 252 includes a surface area 254 for capturing images. In an exemplary implementation, the size of surface area 254 occupies maximum cross sectional area of interior space 204 to facilitate image capture quality. As shown in the example of FIG. 2, the shape (e.g., square or rectangular) of surface 254 can be different from the shape (e.g., circular) of the cross sectional area of interior space 204 (defined for example by the shape of housing 202), in which case, according to an exemplary implementation, the maximum cross sectional area of interior space 204 occupied by image capturing element 250 can be defined by the shape and size of the surface area 254 that fits, for example up to and within the borders of the interior space 204. Optionally, surface area 254 can be reduced or shaped to balance image capture quality and/or illumination and/or laser energy. Further optionally, surface area 254 can be adjusted to ensure stable configuration to be provided by support 260.

According to an optional exemplary implementation, connection 232 extends from proximal portion of image module 250 to maximize amount of interior space remaining for laser fiber 236 and light fiber 234. For example, a harness 280 can be provided to ensure that connection 232 extends proximately from image module 250 essentially parallel to interior of housing 202.

According to another optional exemplary implementation, a plurality of light fibers 234 are configured within all of interior space available outside of image module 250 (or, if provided, support 260) and laser fiber 236.

According to yet another optional exemplary implementation, a lens 270 can be provided with respect to a distal surface of image module 250. Optionally, lens 270 can be fixed or removably attached at distal most portion of distal end 122 of probe 120. Optionally, lens 270 can be attached to, or integrally formed with, image module 250. Different lenses 270 can be attached at distal end 122 in a removable configuration.

In yet another optional implementation, a wireless transceiver (not shown) can be provided as part of image module 250 to wirelessly communicate, for example on-demand or continuously, images captures by image module 250, thereby eliminating wiring 232 and/or providing a redundant communication connection from/to image module 250. Optionally, a wireless transceiver can be provided within hand piece 110 or within probe 120, placed such that the transceiver does not interfere with connections 130.

Other objects, advantages and salient features will become apparent to those skilled in the art from the details provided, which, taken in conjunction with the accompanying drawing figures, describe exemplary embodiments of the disclosure.

While the present disclosure has been shown and described with reference to certain exemplary embodiments thereof, it will be understood by those skilled in the art that various changes in form and details may be made therein without departing from the spirit and scope of the present disclosure and the scope of the claims.

1. An endoscope system comprising:
a probe extending distally from a hand piece, said probe comprising a proximal end and a distal end;
an illumination guide extending from said distal end of said probe to said proximal end of said probe, said illumination guide comprising an illumination guide distal end terminating at a distal end of said probe;
a laser guide extending from said distal end of said probe to said proximal end of said probe, said laser guide comprising a laser guide distal end terminating at said distal end of said probe; and
an image module including an imaging component comprising an electronic image detection module disposed at said distal end of said probe for capturing images at a site of a procedure.

2. The endoscope system of claim 1, wherein the electronic image detection module comprises an image sensor disposed at said distal end of the probe.

3. The endoscope system of claim 1, wherein
the electronic image detection module includes an image sensor disposed at the distal end of the probe,
the image sensor consists of at least one of a CMOS image sensor disposed at the distal end of the probe and a CCD disposed at the distal end of the probe, and
the endoscope system further comprises at least one of:
a wired connection of the image sensor to at least one of an image processing module and an image storage module; and
a wireless connection of the image sensor to the at least one of the image processing module and the image storage module.

4. The endoscope system of claim 1, further comprising a hand piece with the probe extending distally from a distal end of the hand piece.

5. The endoscope system of claim 1, wherein one or more of at least one of optical and electronic connections extend through the probe from the distal end of the probe to at least the proximal end of the probe to facilitate the laser output, the illumination output and connection to the imaging component.

6. The endoscope system of claim 5, wherein the at least one of the optical and electronic connections comprises a laser guide extending from the distal end of the probe to a source of laser energy.

7. The endoscope system of claim 1, further comprising an optical connection, comprising an illumination guide including an optical fiber or a bundle of fibers, extends from the distal end of the probe to a source of illumination.

8. The endoscope system of claim 1, further comprising an electronic connection, comprising one or more conductive wires, extends from the distal end of the probe to at least one of an image processing unit, video processing unit, a video storage unit, and a transmission unit.

9. The endoscope system of claim 1, wherein the distal portion of the probe comprises:
the laser guide comprising a laser fiber disposed within an inner diameter of the distal portion of the probe;
the image module comprising an image sensor, including at least one of a CMOS and a CCD, disposed within the inner diameter of the distal portion of the probe not occupied by the laser fiber; and
the illumination guide comprising an illumination bundle including a plurality of fibers filling the remaining of the inner diameter of the distal portion of the probe not occupied by the laser fiber and the image sensor.

10. The endoscope system of claim 1, wherein said laser guide selectively accommodates input from laser energy sources having different wavelengths including a green laser having a wavelength of 532 nanometers.

11. The endoscope system of claim 1, wherein the probe comprises a housing having an interior space which extends from said distal end to said proximal end of the probe, said image module is disposed at said distal end of said probe, and said image module includes an image capturing element comprising a CMOS imaging chip or a CCD imaging chip.

12. The endoscope system of claim 1, further comprising a support for said image module disposed at said distal end of said probe.

13. The endoscope system of claim 11, wherein the image capturing element includes a surface area for capturing images, the size of the surface area occupies maximum cross sectional area of the interior space.

14. The endoscope system of claim 11, wherein an electronic connections extend from a proximal portion of the image module to maximize amount of the interior space remaining for the laser guide and the illumination guide.

15. The endoscope system of claim 14, further comprising a harness to guide said electronic connection to said image module to extend proximately from said image module essentially parallel to an interior of said housing.

16. The endoscope system of claim 1, wherein said illumination guide comprises a plurality of light fibers configured to occupy all of said interior space outside of said image module and said laser guide.

17. The endoscope system of claim 1, further comprising a lens disposed with respect to a distal surface of said image module.

18. The endoscope system of claim 17, wherein the lens is fixed, or removably attached, at a distal most portion of said distal end of said probe.

19. The endoscope system of claim 1, further comprising a wireless transceiver to wirelessly communicate, on-demand or continuously, images captures by said image module.

20. The endoscope system claim 19, wherein said wireless transceiver is configured within said image module.

21. The endoscope system of claim 2, wherein said image sensor comprises a CMOS (complementary metal-oxide semiconductor) image sensor disposed at said distal end of the probe or a CCD (charge-coupled device) disposed at said distal end of the probe.

22. The endoscope system of claim 4, wherein the proximal endo of the probe is attached to the hand piece and the distal end of the probe is disposed closest to the site of the procedure during operation.

23. The endoscope system of claim 6, wherein the laser guide comprises an optical fiber extending from the distal end of the probe to the source of laser energy.

24. The endoscope system of claim 1, wherein the probe comprises a housing having an interior space which extends from said distal end to said proximal end of the probe, said image module is disposed at said distal end of said probe, and said image module includes an image capturing element consisting of a CMOS imaging chip or a CCD imaging chip.
